(19) United States
(12) Patent Application Publication
(10) Pub. No.: US 2026/0109680 A1
(43) Pub. Date: Apr. 23, 2026

(54) METHOD FOR SYNTHESIZING 2,5-FURANDICARBOXYLIC ACID BY CARBONYLATION OF FUROIC ACID

(71) Applicant: INSTITUTE OF CHEMICAL INDUSTRY OF FOREST PRODUCTS, CAF, Nanjing (CN)

(72) Inventors: Kui WANG, Nanjing (CN); Shanyong WANG, Nanjing (CN); Jianchun JIANG, Nanjing (CN); Jun YE, Nanjing (CN)

(73) Assignee: INSTITUTE OF CHEMICAL INDUSTRY OF FOREST PRODUCTS, CAF, Nanjing (CN)

(21) Appl. No.: 19/477,497

(22) PCT Filed: Sep. 6, 2024
(86) PCT No.: PCT/CN2024/117566

§ 371 Date: Oct. 22, 2025

(30) Foreign Application Priority Data
Oct. 26, 2023 (CN) 202311401320.3

(51) Int. Cl. C07D 307/68

(52) U.S. Cl. CPC C07D 307/68

(57) ABSTRACT

A method for synthesizing 2,5-furandicarboxylic acid by carbonylation of furoic acid is provided. In this method, furoic acid and an organic base are added to a biphasic solvent system. After completion of the reaction, two phases are separated, the organic acid is added to the aqueous phase, and the precipitate is collected by filtration. The precipitate is then washed and dried to obtain the 2,5-furandicarboxylic acid. The biphasic solvent system is a mixture of a basic metal salt solution and a biomass-based polar aprotic solvent, and a CO₂-donating reagent may or may not be added to the biphasic solvent system. The method has simple synthesis and separation and purification processes, high product purity, a high carbonylation reaction rate, reduced raw material costs, and is in line with the development concept of green chemistry.

### CROSS REFERENCE TO THE RELATED APPLICATIONS

This application is the national phase entry of International Application No. PCT/CN2024/117566, filed on Sep. 6, 2024, which is based upon and claims priority to Chinese Patent Application No. 202311401320.3, filed on Oct. 26, 2023, the entire contents of which are incorporated herein by reference.

### TECHNICAL FIELD

This invention belongs to the field of furan-based biomass chemicals and materials, and specifically relates to a method for synthesizing 2,5-furandicarboxylic acid by carbonylation of furoic acid.

### BACKGROUND

2,5-Furandicarboxylic acid (FDCA), a furan-based product with an aromatic structure, shares significant structural and functional similarities with petroleum-based terephthalic acid. Polyethylene 2,5-furandicarboxylate (PEF) prepared from FDCA and ethylene glycol exhibits superior thermodynamic, strength, and barrier properties to polyethylene terephthalate (PET). FDCA is generally considered a green diacid monomer to replace terephthalic acid, thereby driving the release of demand for FDCA. According to a research report released by the New Sijie Industry Research Center, the global FDCA market is expected to grow at a compound annual growth rate of over 8.5% from 2022 to 2027, reaching US$820 million in 2027.

Currently, the synthesis of FDCA mainly involves three pathways: the 5-hydroxymethylfurfural (HMF) oxidation pathway, the glucaric acid pathway, and the furoic acid pathway. The HMF oxidation pathway uses HMF as a substrate and oxidizes it to FDCA via thermal catalytic oxidation or photoelectron catalytic oxidation. The catalyst for this process is typically a precious metal, and large-scale production has been achieved. However, due to the high price of HMF, the production cost of 2,5-furandicarboxylic acid is much higher than that of terephthalic acid, which limits the large-scale production and application of FDCA. The glucaric acid pathway involves selectively oxidizing the C1 and C6 positions of the glucose to carboxyl groups, followed by direct synthesis of 2,5-furandicarboxylic acid under appropriate dehydration conditions. The primary challenge of this pathway is the selective oxidation in the first step, and related theories and technologies are under development.

The furoic acid pathway primarily involves direct carbonylation of furoic acid, bromination of furoic acid, and disproportionation of furoic acid. Since Banerjee et al. (Nature, 2016, 7593:215-219) reported the preparation of FDCA by cesium carbonate-assisted direct carbonylation using CO₂ as a carbonylation reagent and furoic acid as a substrate, research on carbonate-assisted preparation of FDCA from furoic acid has continued to emerge. Chinese Patent CN116283849A provides a method for preparing FDCA using cesium salts as a raw material, and Chinese Patent CN115991686A further prepares a carbonate-supported superbase catalyst to assist the synthesis of FDCA. However, this approach involves a solid-phase heterogeneous reaction with relatively harsh reaction conditions, and related research has not yet made significant progress. There are two typical pathways for obtaining FDCA from furoic acid bromination: direct oxidation and direct synthesis with CO₂ after preparation as a Grignard reagent. Both methods require bromination of furoic acid, which is a major cost-limiting factor. The preparation of FDCA by disproportionation of furoic acid originates from benzene and terephthalic acid produced by mixing benzoic acid with a metal halide salt by Ogata et al. (J. Am. Chem. Soc., 1957, 22:6005-6008). However, the FDCA prepared by this method has many isomers and the reaction conditions are also relatively harsh.

### SUMMARY

### Technical Problem

In response to the aforementioned problems in the prior art, the technical problem to be solved by the present invention is to provide a method for synthesizing 2,5-furandicarboxylic acid by carbonylation of furoic acid, addressing the high cost and harsh reaction conditions currently associated with the production of 2,5-furandicarboxylic acid.

### Technical Solution

To address these issues, the present invention adopts the following technical solutions:

A method for synthesizing 2,5-furandicarboxylic acid by carbonylation of furoic acid, including adding furoic acid and an organic base to a biphasic solvent system; separating two phases after completing a reaction; adding an organic acid to the aqueous phase; and collecting a precipitate by filtration, washing the precipitate, and drying to obtain the 2,5-furandicarboxylic acid; where the biphasic solvent system is a mixture of a basic metal salt solution and a biomass-based polar aprotic solvent, and a CO₂-donating reagent may or may not be added to the biphasic solvent system.

Furthermore, a volume ratio of the basic metal salt solution to the biomass-based polar aprotic solvent is 1-9:1-9.

Furthermore, a basic metal salt is one or a mixture of Na₂CO₃, K₂CO₃, NaOH, and KOH, and a concentration of the basic metal salt solution is 0.5-5 mol/L.

Preferably, the concentration of the basic metal salt solution is 0.5-1.5 mol/L.

Furthermore, the biomass-based polar aprotic solvent is one or a mixture of tetrahydrofuran, acetonitrile, and propylene carbonate.

Furthermore, the organic base is one or more of sodium methoxide, sodium formate, sodium ethoxide, sodium acetate, triphenylacetic acid, and trimethylacetic acid.

Furthermore, a concentration of the furoic acid and a concentration of the organic base in a reaction system are both 0.1-1 mol/L. Preferably, the concentration of the furoic acid in the reaction system is 0.4-0.6 mol/L, and the concentration of the organic base in the reaction system is 0.3-0.5 mol/L.

Furthermore, the CO₂-donating reagent is one or a mixture of triphenylacetic acid, trimethylacetic acid, ethylene carbonate, and propylene carbonate.

Furthermore, the organic acid is one or a mixture of formic acid, acetic acid, and propionic acid.

Furthermore, the specific steps are as follows:
1) dissolving a basic metal salt in water to obtain the basic metal salt solution, and then mixing the basic metal salt solution with the biomass-based polar aprotic solvent to construct the biphasic solvent system;
2) adding the furoic acid and the organic base to the biphasic solvent system constructed in the step 1), with or without an addition of the CO₂-donating reagent, and performing a reaction at 140-220° C. with continuous stirring for 1-12 h;
3) after completion of the reaction, separating the aqueous and organic phases of a reaction system in the step 2), subjecting the organic phase to reduced pressure distillation at 40-80° C. to recover, adding the organic acid dropwise to the aqueous phase until no further precipitation occurs, and collecting the precipitate by filtration to obtain crude 2,5-furandicarboxylic acid; and
4) continuously washing the crude 2,5-furandicarboxylic acid in a washing solution until a conductivity of the washing solution does not change significantly, and then drying at room temperature to obtain the 2,5-furandicarboxylic acid.

Furthermore, in the step 4), the washing solution is an organic acid-alcohol solution, the organic acid is the same as the organic acid used in the step 3), the alcohol is methanol or ethanol, and a volume ratio of the acid to the alcohol is 5-9:1-6. Preferably, the volume ratio of the acid to the alcohol is 5-8:2-5.

Preferably, a reaction temperature in the step 2) is 160-200° C., and a reaction time is 3-6 h.

Preferably, a distillation temperature in the step 3) is 60-70° C.

The schematic diagram of the conversion of the furoic acid to the 2,5-furandicarboxylic acid in a biphasic system of the present invention is as follows:

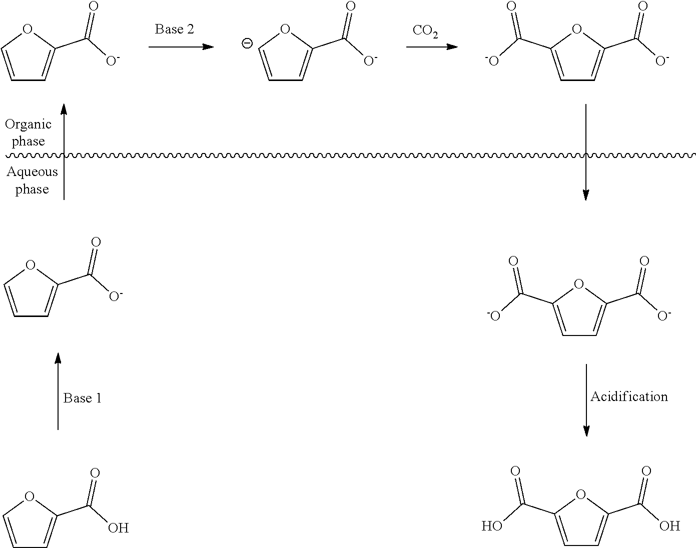

Where, base 1 is the basic metal salt; base 2 is the organic base.

### Beneficial Effects

Beneficial effects: Compared with the prior art, the advantages of the present invention are:
(1) The basic metal salt used in the present invention can simultaneously serve as a base and a phase separation salt, promoting the formation of a biphasic system and assisting the deprotonation of the furoic acid; the biomass polar aprotic solvent can simultaneously serve as a solvent and a CO₂-donating reagent.
(2) The organic base that promotes the carbonylation reaction in the present invention decomposes to generate CO₂ and a superbase at a specific temperature, thereby increasing the carbonylation reaction rate. After the FDCA is formed, the presence of the basic carbonate promotes its transfer to the aqueous phase, which is beneficial to the separation of the product.
(3) The solvent of the present invention, as an inducing agent for CO₂ activation, significantly reduces the reaction energy barrier of the carbonylation process, showing that the reaction temperature range (160-200° C.) is significantly lower than the current direct carbonylation reaction temperature of furoic acid (240-300° C.), and the total pressure of the reaction system (0-0.3 MPa) is also lower than the currently reported furoic acid carbonylation process (3-10 MPa), and no superbase compound is used in the process.
(4) The biphasic solvent system used in the present invention not only serves as a solvent but also has a catalytic effect, thereby increasing the reaction rate.

### BRIEF DESCRIPTION OF THE DRAWINGS

FIG. 1 is a proton nuclear magnetic resonance (H-NMR) (a) spectrum of FDCA prepared according to the present invention;

FIG. 2 is a carbon-13 nuclear magnetic resonance (C-NMR) (b) spectrum of FDCA prepared according to the present invention.

### DETAILED DESCRIPTION OF THE EMBODIMENTS

The present invention will be further described below with reference to specific examples.

The yield, purity, and conversion of FDCA prepared in the following examples were calculated according to the following formula:

0.1 g of the products before and after purification were evenly placed in Petri dishes that have been brought to constant weight, covered with a dust-free paper, and then placed in an oven at 105° C. for 6 h until constant weight was achieved. The sample dryness was determined. The yield Y_{FDCA} of purified FDCA was calculated as follows:

$${Y}_{FDCA} = \frac{{n}_{FDCA}}{{n}_{acid 1}^{Furoic}}$$

where n_{FDCA} is the amount of substance of the product FDCA, and n_{furoic acid 1} is the amount of substance of the reaction substrate furoic acid.

The purity P_{FDCA} of the FDCA was calculated as follows:

$${P}_{FDCA} = \frac{{m}_{FDCA 1}}{{m}_{FDCA 2}}$$

where m_{FDCA 1} is the mass calculated by liquid chromatography, and m_{FDCA 2} is the mass measured.

The conversion C_{FDCA} of the FDCA was calculated as follows:

$${C}_{FDCA} = \frac{{n}_{FDCA}}{{n}_{acid 2}^{Furoic}}$$

where n_{FDCA} is the amount of substance of the product FDCA, and n_{furoic acid 2} is the amount of substance of the furoic acid reacted.

The purity of FDCA prepared in the following examples was determined by chromatographic purity according to the following procedure:

0.1 g (absolute dry equivalent) of the product was placed in a beaker and dissolved in an appropriate amount of hot water. The solution was then cooled to room temperature and diluted to 500 mL. The solution was filtered through a 0.22 μm organic syringe filter and used to determine the concentration of the FDCA. Analysis was performed using a liquid chromatograph (Watere 2695) equipped with a differential refractive index detector (2414 RI Detector) and an HPX-87H organic acid analysis column. The conditions were: a flow rate of 0.6 mL/min, a column temperature of 55° C., a detector temperature of 40° C., and a mobile phase of 5 mM H₂SO₄. The actual chromatographic concentration of FDCA in the dissolved sample was obtained by referring to a pre-drawn FDCA standard curve, and the chromatographic purity of the sample is calculated based on this concentration.

### Example 1

The 1 mol/L aqueous solution of Na₂CO₃ was prepared and mixed with the propylene carbonate in the 1:9 (v:v) ratio to form the biphasic solvent system. The propylene carbonate also provided CO₂. The furoic acid and the triphenylacetic acid were then weighed and added to the biphasic solvent system. The concentrations of the furoic acid and the triphenylacetic acid in the total solvent were each 0.4 mol/L (the solutes were uniformly dissolved in each phase). The reaction vessel was placed at 160° C. for a reaction with continuous stirring for 6 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the methanol and the formic acid in the volume ratio of 8:2) until the conductivity of the washing solution did not change significantly. Finally, the washed FDCA was dried at room temperature to obtain the FDCA.

### Example 2

The 1 mol/L aqueous solution of Na₂CO₃ was prepared and mixed with the acetonitrile in the 3:7 (v:v) ratio to form the biphasic solvent system. The furoic acid and the sodium acetate were then weighed and added to the biphasic solvent system. The concentrations of the furoic acid and the sodium acetate in the total solvent were each 0.4 mol/L (the solutes were uniformly dissolved in each phase). The propylene carbonate, 10% of the total solvent volume, was then added to the reaction vessel. The reaction vessel was placed at 160° C. for a reaction with continuous stirring for 4 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the ethanol and the formic acid in the volume ratio of 7:3) until the conductivity of the washing solution did not change significantly. Finally, the washed FDCA was dried at room temperature to obtain the FDCA.

### Example 3

The 1 mol/L aqueous solution of Na₂CO₃ was prepared and mixed with the tetrahydrofuran in the 3:7 (v:v) ratio to form the biphasic solvent system. The furoic acid and the triphenylacetic acid were then weighed and added to the biphasic solvent system. The concentrations of the furoic acid and the triphenylacetic acid in the total solvent were each 0.4 mol/L (the solutes were uniformly dissolved in each phase). The propylene carbonate, 10% of the total solvent volume, was then added to the reaction vessel. The reaction vessel was placed at 160° C. for a reaction with continuous stirring for 6 h. After completion of the reaction, the aqueous and organic phases were separated using the separatory funnel. The organic phase was subjected to the reduced pressure distillation at 60° C. using the rotary evaporator to recover the solvent. The acetic acid was added dropwise to the aqueous phase until no further precipitation occurred. The precipitate was collected by filtration to obtain the crude FDCA. The crude FDCA was then washed multiple times with the acetic acid-alcohol solution (with the methanol and the acetic acid in the volume ratio of 6:4) until the conductivity of the washing solution did not change significantly. Finally, the washed FDCA was dried at room temperature to obtain the FDCA.

### Example 4

The 1 mol/L aqueous solution of Na₂CO₃ was prepared and mixed with the acetonitrile in the 2:8 (v:v) ratio to form the biphasic solvent system. The furoic acid and the triphenylacetic acid were then weighed and added to the biphasic solvent system. The concentrations of the furoic acid and the triphenylacetic acid in the total solvent were 0.4 mol/L and 0.6 mol/L, respectively (the solutes were uniformly dissolved in each phase). The propylene carbonate, 10% of the total solvent volume, was then added to the reaction vessel. The reaction vessel was placed at 180° C. for a reaction with continuous stirring for 4 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the methanol and the formic acid in the volume ratio of 6:4) until the conductivity of the washing solution did not change significantly. Finally, the washed FDCA was dried at room temperature to obtain the FDCA.

### Example 5

The 1 mol/L aqueous solution of K₂CO₃ was prepared and mixed with the tetrahydrofuran in the 3:7 (v:v) ratio to form the biphasic solvent system. The furoic acid and the trimethylacetic acid were then weighed and added to the biphasic solvent system. The concentrations of the furoic acid and the trimethylacetic acid in the total solvent were 0.4 mol/L and 0.8 mol/L, respectively (the solutes were uniformly dissolved in each phase). The propylene carbonate, 10% of the total solvent volume, was then added to the reaction vessel. The reaction vessel was placed at 180° C. for a reaction with continuous stirring for 6 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the ethanol and the formic acid in the volume ratio of 7:3) until the conductivity of the washing solution did not change significantly. Finally, the washed FDCA was dried at room temperature to obtain the FDCA.

### Example 6

The 1 mol/L aqueous solution of K₂CO₃ was prepared and mixed with the tetrahydrofuran in the 2:8 (v:v) ratio to form the biphasic solvent system. The furoic acid and the sodium acetate were then weighed and added to the biphasic solvent system. The concentrations of the furoic acid and the sodium acetate in the total solvent were each 0.8 mol/L (the solutes were uniformly dissolved in each phase). The propylene carbonate, 10% of the total solvent volume, was then added to the reaction vessel. The reaction vessel was placed at 200° C. for a reaction with continuous stirring for 6 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the methanol and the formic acid in the volume ratio of 8:2) until the conductivity of the washing solution did not change significantly. Finally, the washed FDCA was dried at room temperature to obtain FDCA.

### Example 7

The 1 mol/L aqueous solution of NaOH was prepared and mixed with the propylene carbonate in the 3:7 (v:v) ratio to form the biphasic solvent system. The propylene carbonate also provided CO₂. The furoic acid and the sodium acetate were then weighed and added to the biphasic solvent system. The concentrations of the furoic acid and the sodium acetate in the total solvent were each 0.6 mol/L (the solutes were uniformly dissolved in each phase). The reaction vessel was placed at 200° C. for a reaction with continuous stirring for 6 h. After completion of the reaction, the aqueous and organic phases were separated using the separatory funnel. The organic phase was subjected to reduced pressure distillation at 60° C. using the rotary evaporator to recover the solvent. The acetic acid was added dropwise to the aqueous phase until no further precipitation occurred. The precipitate was collected by filtration to obtain crude FDCA. The crude FDCA was then washed multiple times with the acetic acid-alcohol solution (with the ethanol and the acetic acid in the volume ratio of 8:2) until the conductivity of the washing solution did not change significantly. Finally, the washed FDCA was dried at room temperature to obtain the FDCA.

### Example 8

The 1 mol/L aqueous solution of NaOH was prepared and mixed with the acetonitrile in the 3:7 (v:v) ratio to form the biphasic solvent system. The furoic acid and the triphenylacetic acid were then weighed and added to the biphasic solvent system. The concentration of the furoic acid and the triphenylacetic acid in the total solvent were each 0.4 mol/L (the solutes were uniformly dissolved in each phase). The propylene carbonate, 10% of the total solvent volume, was then added to the reaction vessel. The reaction vessel was placed at 200° C. for a reaction with continuous stirring for 4 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the ethanol and the formic acid in the volume ratio of 8:2) until the conductivity of the washing solution did not change significantly. Finally, the washed FDCA was dried at room temperature to obtain the FDCA.

### Example 9

The 1 mol/L aqueous solution of KOH was prepared and mixed with the acetonitrile in the 2:8 (v:v) ratio to form the biphasic solvent system. The furoic acid and the trimethylacetic acid were then weighed and added to the biphasic solvent system. The concentration of the furoic acid and the trimethylacetic acid in the total solvent were 0.4 mol/L and 0.6 mol/L, respectively (the solutes were uniformly dissolved in each phase). The trimethylacetic acid also provided CO₂. The reaction vessel was placed at 180° C. for a reaction with continuous stirring for 6 h. After completion of the reaction, the aqueous and organic phases were separated using the separatory funnel. The organic phase was subjected to the reduced pressure distillation at 60° C. using the rotary evaporator to recover the solvent. The acetic acid was added dropwise to the aqueous phase until no further precipitation occurred. The precipitate was collected by filtration to obtain the crude FDCA. The crude FDCA was then washed multiple times with the acetic acid-alcohol solution (with the methanol and the acetic acid in the volume ratio of 7:3) until the conductivity of the washing solution did not change significantly. Finally, the washed FDCA was dried at room temperature to obtain the FDCA.

### Example 10

The 1 mol/L aqueous solution of KOH was prepared and mixed with the acetonitrile in the 4:6 (v:v) ratio to form the biphasic solvent system. The furoic acid and the sodium ethoxide were then weighed and added to the biphasic solvent system. The concentrations of the furoic acid and the sodium ethoxide in the total solvent were 0.4 mol/L and 0.8 mol/L, respectively (the solutes were uniformly dissolved in each phase). The propylene carbonate, 10% of the total solvent volume, was then added to the reaction vessel. The reaction vessel was placed at 160° C. for a reaction with continuous stirring for 4 h. After completion of the reaction, the aqueous and organic phases were separated using the separatory funnel. The organic phase was subjected to the reduced pressure distillation at 60° C. using the rotary evaporator to recover the solvent. The acetic acid was added dropwise to the aqueous phase until no further precipitation occurred. The precipitate was collected by filtration to obtain the crude FDCA. The crude FDCA was then washed multiple times with the acetic acid-alcohol solution (with the methanol and the acetic acid in the volume ratio of 7:3) until the conductivity of the washing solution did not change significantly. Finally, the washed FDCA was dried at room temperature to obtain the FDCA.

### Example 11

The 0.5 mol/L aqueous solution of K₂CO₃ was prepared and mixed with the acetonitrile in the 3:7 (v:v) ratio to form the biphasic solvent system. The furoic acid and the trimethylacetic acid were then weighed and added to the biphasic solvent system. The concentrations of the furoic acid and the trimethylacetic acid in the total solvent were 0.4 mol/L and 0.8 mol/L, respectively (the solutes were uniformly dissolved in each phase). The propylene carbonate, 10% of the total solvent volume, was then added to the reaction vessel. The reaction vessel was placed at 180° C. for a reaction with continuous stirring for 6 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the ethanol and the formic acid in the volume ratio of 8:2) until the conductivity of the washing solution did not change significantly. Finally, the washed FDCA was dried at room temperature to obtain the FDCA.

### Example 12

The 0.5 mol/L aqueous solution of the NaOH was prepared and mixed with the acetonitrile in the 2:8 (v:v) ratio to form the biphasic solvent system. The furoic acid and the triphenylacetic acid were then weighed and added to the biphasic solvent system. The concentrations of the furoic acid and the triphenylacetic acid in the total solvent were each 0.8 mol/L (the solutes were uniformly dissolved in all phases). The propylene carbonate, 10% of the total solvent volume, was then added to the reaction vessel. The reaction vessel was placed at 160° C. for a reaction with continuous stirring for 4 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the ethanol and the formic acid in the volume ratio of 8:2) until the conductivity of the washing solution did not change significantly. Finally, the washed FDCA was dried at room temperature to obtain the FDCA.

### Example 13

The 1 mol/L aqueous solution of NaCl was prepared and mixed with the N,N-dimethylformamide in the 2:8 (v:v) ratio to form the biphasic solvent system. The furoic acid and the sodium acetate were then weighed and added to the biphasic solvent system. The concentrations of the furoic acid and the sodium acetate in the total solvent were each 0.8 mol/L (the solutes were uniformly dissolved in each phase). The propylene carbonate, 10% of the total solvent volume, was then added to the reaction vessel. The reaction vessel was placed at 180° C. for a reaction with continuous stirring for 4 h. After completion of the reaction, the aqueous and organic phases were separated using the separatory funnel. The organic phase was subjected to the reduced pressure distillation at 60° C. using the rotary evaporator to recover the solvent. The formic acid was added dropwise to the aqueous phase until no further precipitation occurred. The precipitate was collected by filtration to obtain the crude FDCA. The crude FDCA was then washed multiple times with the formic acid-alcohol solution (with the ethanol and the formic acid in the volume ratio of 8:2) until the conductivity of the washing solution did not change significantly. Finally, the washed FDCA was dried at room temperature to obtain the FDCA.

The product yields and product conversion rates of the FDCA prepared in Examples 1-13 of the present invention are shown in Table 1, below.

Table 1. Product yields and product conversion rates of the FDCA prepared in Examples 1-13

| | Conversion | | |
|---|---|---|---|
| | rate of the | Yield of | Yield of |
| | furoic | the crude | the purified |
| | acid (mol %) | FDCA (mol %) | FDCA (mol %) |
| Example 1 | 28.95 | 23.16 | 16.68 |
| Example 2 | 97.91 | 83.23 | 72.41 |
| Example 3 | 5.08 | 4.12 | 3.13 |
| Example 4 | 98.54 | 91.64 | 61.40 |
| Example 5 | 23.81 | 17.27 | 15.02 |
| Example 6 | 21.08 | 15.21 | 10.81 |
| Example 7 | 31.40 | 25.64 | 24.36 |
| Example 8 | 85.83 | 74.34 | 68.54 |
| Example 9 | 82.60 | 67.28 | 48.91 |
| Example 10 | 68.33 | 54.33 | 38.95 |
| Example 11 | 99.17 | 90.89 | 82.89 |
| Example 12 | 65.98 | 56.17 | 51.68 |
| Example 13 | 10.61 | — | — |

The FDCA prepared in Examples 1-12 of the present invention had the purity of ≥99%. Due to the selected solvent, the yield of FDCA in Example 13 could not be determined.

Table 1 shows the product yields and product conversion rates of the FDCA prepared in Examples 1-13. As shown in the table, the solvent type is crucial for the catalytic synthesis of FDCA. The ratio of mixed solvents, temperature, reaction time, and the concentration of the base and basic salt also have the significant correlation with the purity of FDCA. The mixed acid-alcohol washing solution effectively removes salts and other impurities from the product while simultaneously ensuring minimal dissolution of the FDCA, thereby ensuring that the purified FDCA has relatively high yield while maintaining high purity.

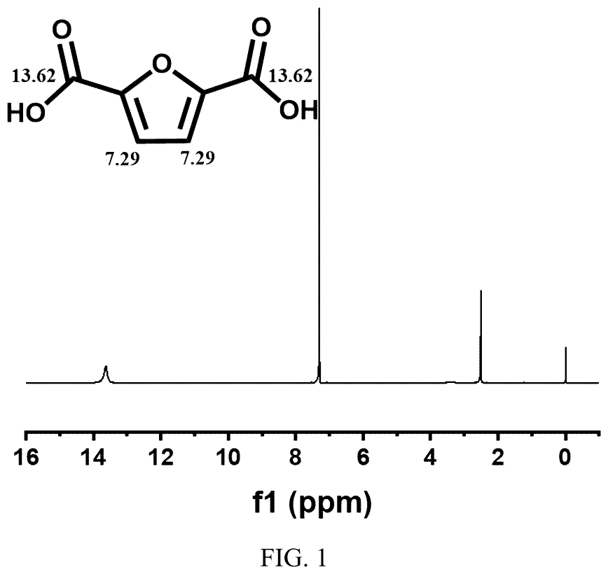

FIG. 1 shows the H-NMR (a) spectrum of the FDCA prepared in the present invention. The characteristic shifts of the H atoms on the FDCA appear at 7.29 ppm and 13.62 ppm, respectively. No other significant chemical shift peaks were observed, indicating that the purified product is relatively pure.

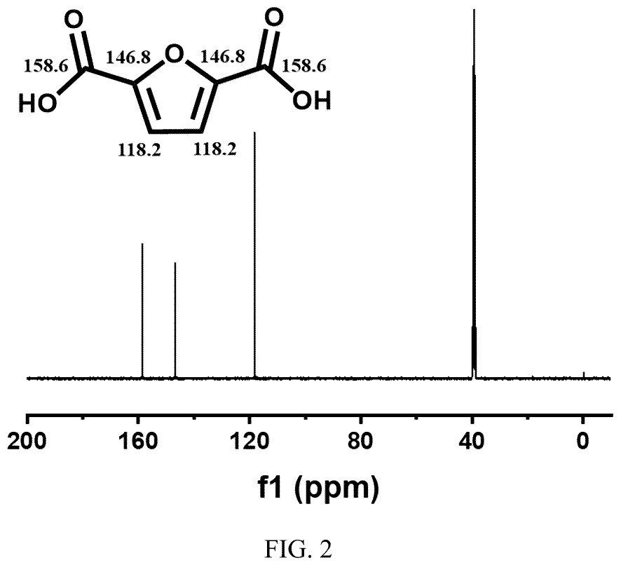

FIG. 2 shows the C-NMR (b) spectrum of the FDCA prepared in the present invention. As can be seen from the figure, the characteristic shifts of the C atoms on the FDCA appear at 118.2 ppm, 146.8 ppm, and 158.6 ppm, respectively, and no other significant chemical shift peaks are observed, indicating that the purified product is relatively pure.

What is claimed is:

1-10. (canceled)

11. A method for synthesizing 2,5-furandicarboxylic acid by carbonylation of furoic acid, comprising adding the furoic acid and an organic base to a biphasic solvent system; separating an aqueous phase and an organic phase after completing a reaction; adding an organic acid to the aqueous phase; and collecting a precipitate by filtration, washing the precipitate, and drying to obtain the 2,5-furandicarboxylic acid; wherein the biphasic solvent system is a mixture of a basic metal salt solution and a biomass-based polar aprotic solvent, and a basic metal salt is one or a mixture of Na₂CO₃, K₂CO₃, NaOH, and KOH; the biomass-based polar aprotic solvent is acetonitrile; the organic base is one or more of sodium acetate, triphenylacetic acid, and trimethylacetic acid; a CO₂-donating reagent is added to the biphasic solvent system; and the CO₂-donating reagent is one or a mixture of ethylene carbonate and propylene carbonate.

12. The method for synthesizing the 2,5-furandicarboxylic acid by the carbonylation of the furoic acid according to claim 11, wherein a volume ratio of the basic metal salt solution to the biomass-based polar aprotic solvent is 1-9:1-9.

13. The method for synthesizing the 2,5-furandicarboxylic acid by the carbonylation of the furoic acid according to claim 11, wherein a concentration of the basic metal salt solution is 0.5-5 mol/L.

14. The method for synthesizing the 2,5-furandicarboxylic acid by the carbonylation of the furoic acid according to claim 11, wherein a concentration of the furoic acid and a concentration of the organic base in a reaction system are both 0.1-1 mol/L.

15. The method for synthesizing the 2,5-furandicarboxylic acid by the carbonylation of the furoic acid according to claim 11, wherein the organic acid is one or a mixture of formic acid, acetic acid, and propionic acid.

16. The method for synthesizing the 2,5-furandicarboxylic acid by the carbonylation of the furoic acid according to claim 11, wherein steps are as follows:
1) dissolving the basic metal salt in water to obtain the basic metal salt solution, and then mixing the basic metal salt solution with the biomass-based polar aprotic solvent to construct the biphasic solvent system;
2) adding the furoic acid and the organic base to the biphasic solvent system constructed in the step 1), with or without an addition of the CO₂-donating reagent, and performing the reaction at 140-220° C. with continuous stirring for 1-12 h;
3) after completion of the reaction, separating the aqueous phase and the organic phase of a reaction system in the step 2), subjecting the organic phase to reduced pressure distillation at 40-80° C. to recover, adding the organic acid dropwise to the aqueous phase until no further precipitation occurs, and collecting the precipitate by the filtration to obtain crude 2,5-furandicarboxylic acid; and
4) continuously washing the crude 2,5-furandicarboxylic acid in a washing solution until a conductivity of the washing solution does not change significantly, and then drying at room temperature to obtain the 2,5-furandicarboxylic acid.

17. The method for synthesizing the 2,5-furandicarboxylic acid by the carbonylation of the furoic acid according to claim 16, wherein in the step 4), the washing solution is an organic acid-alcohol solution, an organic acid of the organic acid-alcohol solution is the same as the organic acid used in the step 3), an alcohol of the organic acid-alcohol solution is methanol or ethanol, and a volume ratio of the organic acid of the organic acid-alcohol solution to the alcohol of the organic acid-alcohol solution is 5-9:1-6.

18. The method for synthesizing the 2,5-furandicarboxylic acid by the carbonylation of the furoic acid according to claim 12, wherein a concentration of the basic metal salt solution is 0.5-5 mol/L.
